(19) United States
(12) Patent Application Publication
(10) Pub. No.: US 2026/0165853 A1
(43) Pub. Date: Jun. 18, 2026

(54) KNEE JOINT CUSHIONING ADJUSTMENT STRUCTURE

(71) Applicant: Ken Dall Enterprise Co., Ltd., New Taipei City (TW)

(72) Inventors: Chia-Pao CHENG, New Taipei City (TW); Hsiang-Ming WU, New Taipei City (TW); Chih-Hsuan LIANG, New Taipei City (TW)

(21) Appl. No.: 18/979,622

(22) Filed: Dec. 13, 2024

(51) Int. Cl. A61F 2/64; A61F 2/68; A61F 2/50

(52) U.S. Cl. CPC A61F 2/64; A61F 2/68; A61F 2002/5016

(57) ABSTRACT

A knee joint cushioning adjustment structure includes a knee top seat having a first connecting hole, a hydraulic cylinder having second and third connecting holes in upper and lower parts and a link-bar through hole between the second and third connecting holes, and a cushion body. The hydraulic cylinder includes a hydraulic chamber having a piston assembly therein and defining upper and lower fluid compartments, such that during frontward/backward tilting, the knee top seat drives the lower/upper fluid compartment to operate in combination with a frontward-tilting/backward-tilting hydraulic circuit to implement cushioning. The cushion body has a cushion-body through hole in an upper side to receive a push bard of the piston assembly to dispose therein and also has a cushion-body pin hole in a lateral side to receive a fixing pin to penetrate therein to constrain rotation of the push bar for defining a safety cushioning angle for frontward/backward tilting.

### BACKGROUND OF THE INVENTION

### (a) Technical Field of the Invention

The present invention generally relates to the field of knee joints, and more particularly to a cushioning adjustment structure of a knee joint.

### (b) Description of the Prior Art

Knee joint related cushioning adjustment technology has been constantly developing and progressing, such as Chinese Patent CN112334095B, which discloses a valve and a prosthetic knee joint comprising the valve, primarily featuring that the valve has an inlet opening, a first outlet opening, and a second outlet opening, and the valve body is movable, along a moving direction, to a first position and a second position, and first fluid connection extends from the inlet opening to the first outlet opening and second fluid connection extends from the inlet opening to the second outlet opening, and a hydraulic assembly includes one or more fluid channels allowing fluid exiting from the first outlet opening and the second outlet opening to flow into an expansion chamber, wherein the innovation lies in the construction and arrangement of the inlet opening are made such that when the valve body is in the first position, the fluid can exert a resultant force on the valve body, and at least the force in the vertical movement direction acts upward, and when in different positions, the valve body can control the first fluid connection. As such, the sensitivity and reliability of the entire knee joint are improved to a certain extent.

However, referring to the prosthetic knee joint disclosed in Chinese Patent CN105873546B, the main structure is composed of an upper part, a lower part, a fixation device, and a four-part joint system, primarily using the joint system to realize more flexibility, and using orienting achieved with a spring force to realize capability of bending resistance.

To sum up, it is believed that improvements aiming at taking easing control of the movement of the valve body for restricting the fluid, in combination with improving the structural restriction on relative joint bending, can be made to provide better flexibility, and this is the goal to which the present invention is made for.

### SUMMARY OF THE INVENTION

The primary objective of the present invention is to provide a standing cushioning adjustment structure of a knee joint, which comprises: a knee top seat, a hydraulic cylinder, and a cushion body, the knee top seat being formed with a first connecting hole, the hydraulic cylinder comprises an upper part formed with a second connecting hole and a lower part formed with a third connecting hole, and a link-bar through hole formed in a location between the second connecting hole and the third connecting hole, the first connecting hole being arranged at an inner side of the second connecting hole for connection therewith, the hydraulic cylinder being formed, in an interior thereof, with a hydraulic chamber, in which a piston assembly is arranged to extend through a hydraulic cylinder cap to define an upper fluid compartment and a lower fluid compartment, wherein during frontward tilting, the knee top seat drives the lower fluid compartment of the hydraulic cylinder to operate in combination with a frontward-tilting hydraulic circuit to implement cushioning, and during backward tilting, the knee top seat drives the upper fluid compartment of the hydraulic cylinder to operate in combination with the backward-tilting hydraulic circuit to implement cushioning, the cushion body being formed, in an upper side thereof, with a cushion-body through hole to receive a push bar connected to a bottom of the piston assembly to dispose therein, the cushion body being formed, in a lateral side thereof, with a cushion-body pin hole to receive a fixing pin to penetrate therein and constrain the push bar, so as to provide, when mounted with the hydraulic cylinder and the push bar, a cushioning range of 5 degrees for frontward tilting and backward tilting to make the effect of cushioning and shock absorbing more significant.

Compared with the known knee joint cushioning adjustment structures, the present invention exhibits the following advantages:

(1) The range of cushioning is wider, because the push bar and the cushion body are arranged between the knee top seat and the hydraulic cylinder to allow the cushioning range to fulfill an arrangement for 5 degrees for both frontward tilting and backward tilting, and this is considered more dexterous as compared to the conventional arrangement of cushioning in only one direction; (2) the effect of cushioning is better, because the hydraulic cylinder is provided, on a lateral side thereof, with the valve needles, which are capable of adjusting, through rotation thereof, the flowrates of the hydraulic circuits through which fluid flows, the strength of cushioning can be effectively adjusted in both frontward tilting and backward tilting, and each hydraulic circuit is provided with a one-way valve, which is operable, in combination with the above arrangement for flowrate of the hydraulic circuit, to keep the relative flowing direction in the hydraulic circuit, making it more stable and eliminating reversed flowing resulting from pressure; (3) the structural arrangement is more compact, because in addition to the interior of the hydraulic cylinder, the acting force is also transmitted by the mechanism that assists in cushioning through the extension of the push bar into the cushion body at the bottom side, the entire structure is relatively compact, and this also makes the response of the entirety of the knee joint to cushioning faster.

### BRIEF DESCRIPTION OF THE DRAWINGS

FIG. 1 is an exploded view showing the present invention.

FIG. 2 is a schematic view showing an initial state of the present invention.

FIG. 3 is a schematic view showing a frontward-tilting state of the present invention.

FIG. 4 is a schematic view illustrating a frontward-tilting hydraulic circuit of the present invention shown in FIG. 3.

FIG. 5 is a schematic view showing a backward-tilting state of the present invention.

FIG. 6 is a schematic view illustrating a backward-tilting hydraulic circuit of the present invention shown in FIG. 5.

### DETAILED DESCRIPTION OF THE PREFERRED EMBODIMENTS

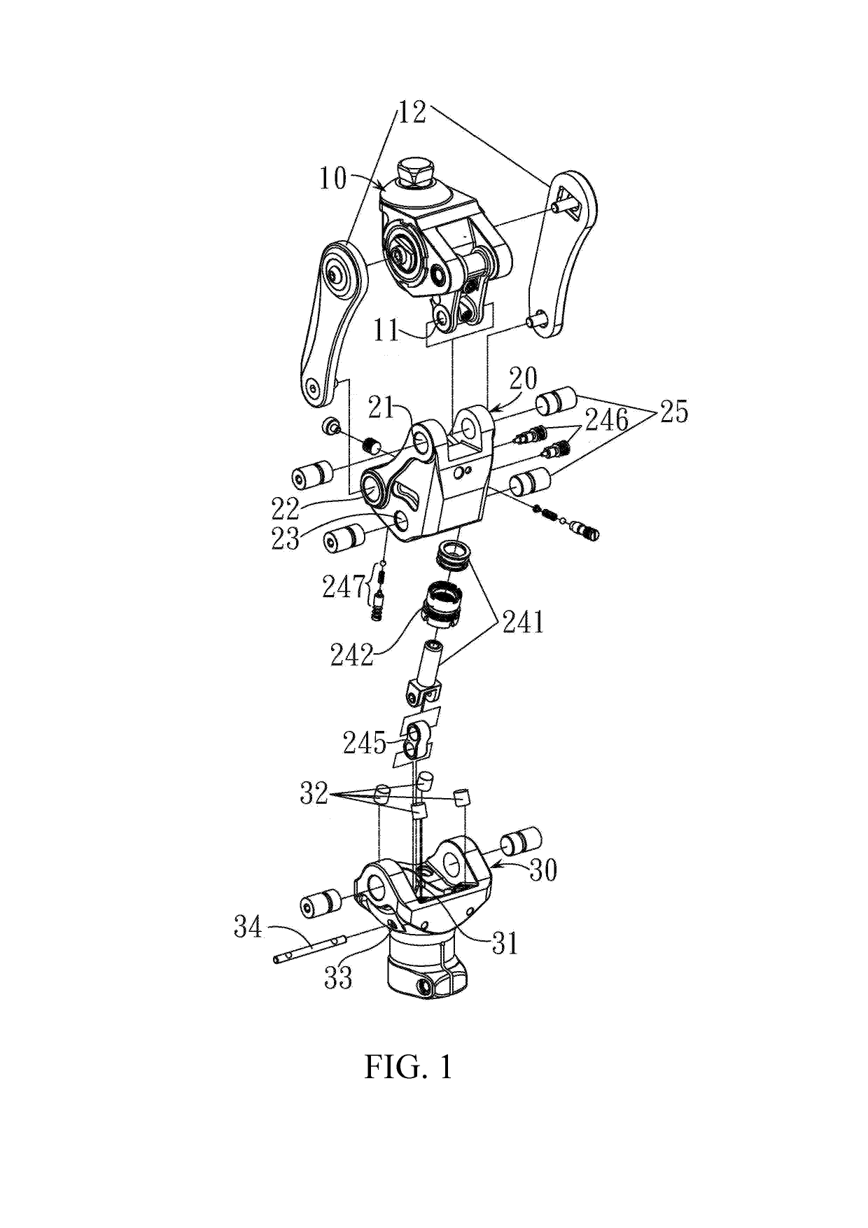

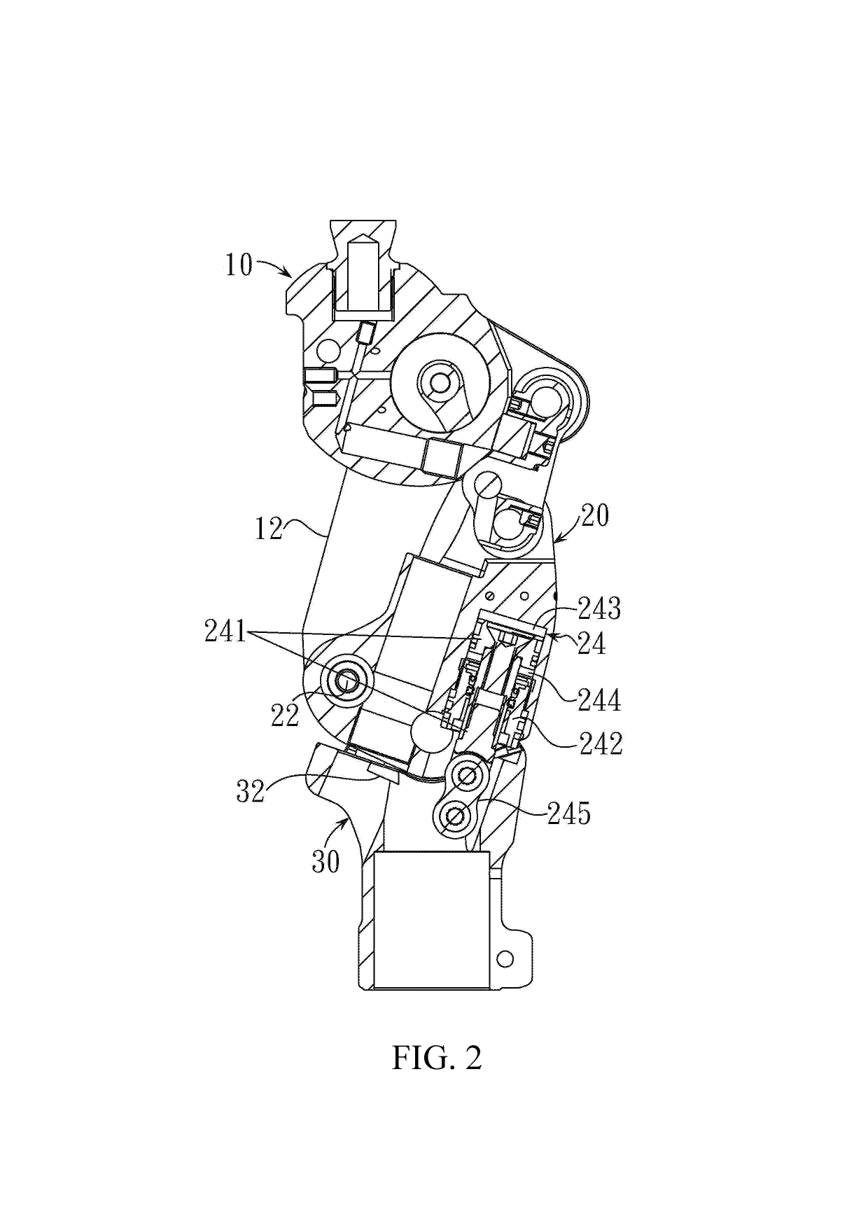

A preferred feasible embodiment generally according to the present invention will be described in detail with reference to FIGS. 1-6 of the drawings in order to increase understanding of the present invention. The present invention relates to a standing cushioning adjustment structure of a knee joint, which comprises: a knee top seat (10), which comprises a first connecting hole (11) and a link bar (12); a hydraulic cylinder (20), which has an upper part comprising a second connecting hole (21) and a lower part comprising a third connecting hole (23), and a link-bar through hole (22) formed between the second connecting hole (21) and the third connecting hole (23). The first connecting hole (11) is positionable at an inner side of the second connecting hole (21) for mating and connecting therewith. The means of connecting mentioned herein refers to any means of fixing commonly known in the field. Details concerning penetrating and fixing by a pair of fixing axles (25) will not be repeatedly discussed hereinafter, while the description is made only with respect to connecting implemented with the fixing axles (25), and the drawings also do not mark the same contents for avoiding confusion. The link bar (12) is connected to the link-bar through hole (22). The hydraulic cylinder (20) is provided, in an interior thereof, with a hydraulic chamber (24), and a piston assembly (241) is arranged inside the hydraulic chamber (21) and extends through a hydraulic cylinder cap (242) to define an upper fluid compartment (243) and a lower fluid compartment (244).

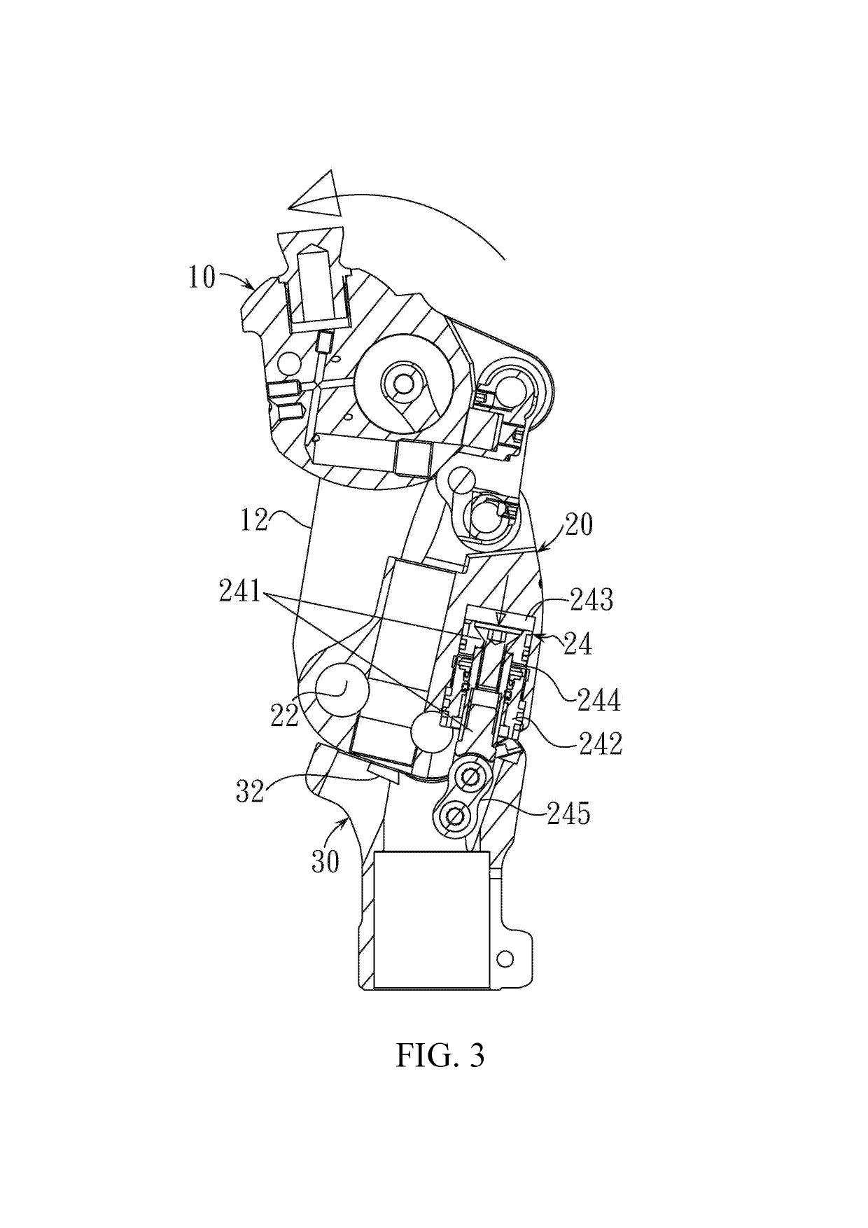

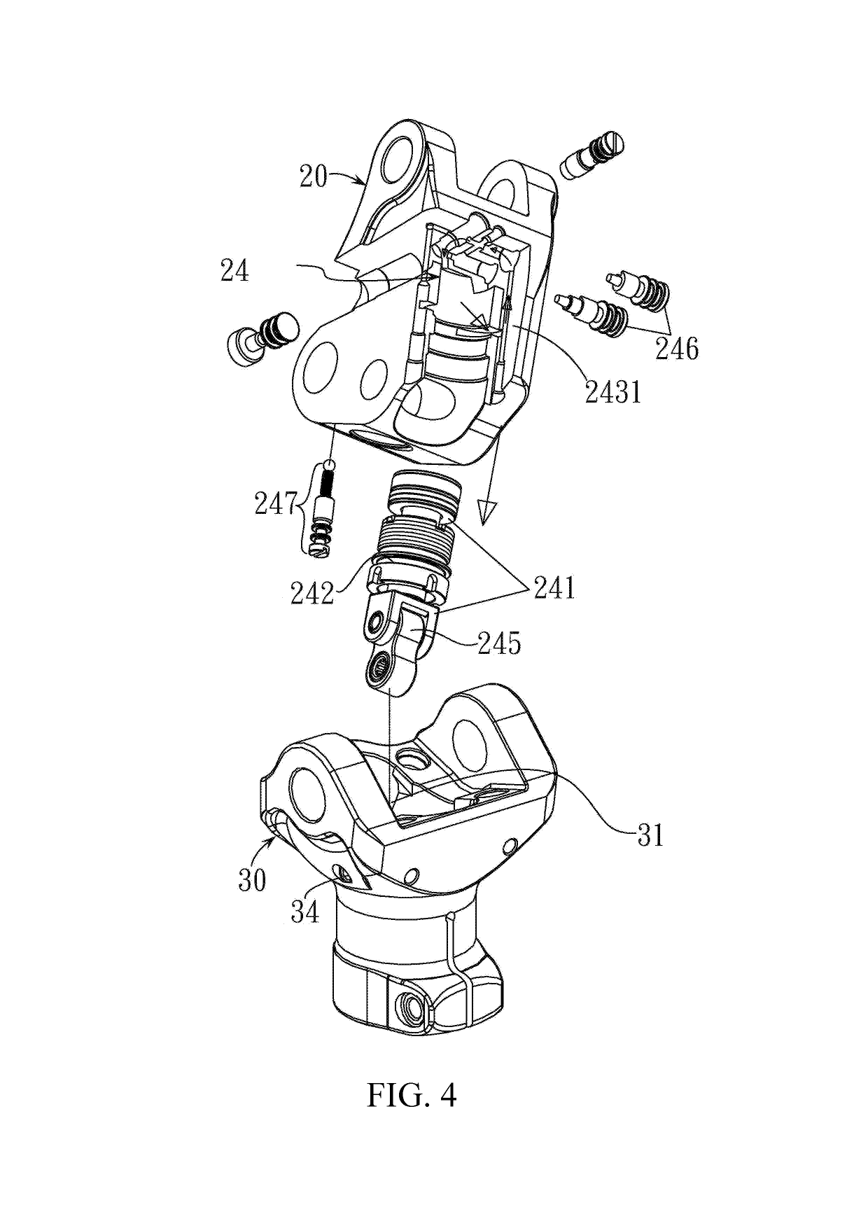

Referring to FIGS. 3-4, when the knee top seat (10) performs a frontward tilting operation, fluid in the lower fluid compartment (244) inside the hydraulic cylinder (20) is driven to follow a frontward-tilting hydraulic circuit (2431) for flowing as indicated by the arrow, so as to cushion the acting force generated by the frontward tilting operation.

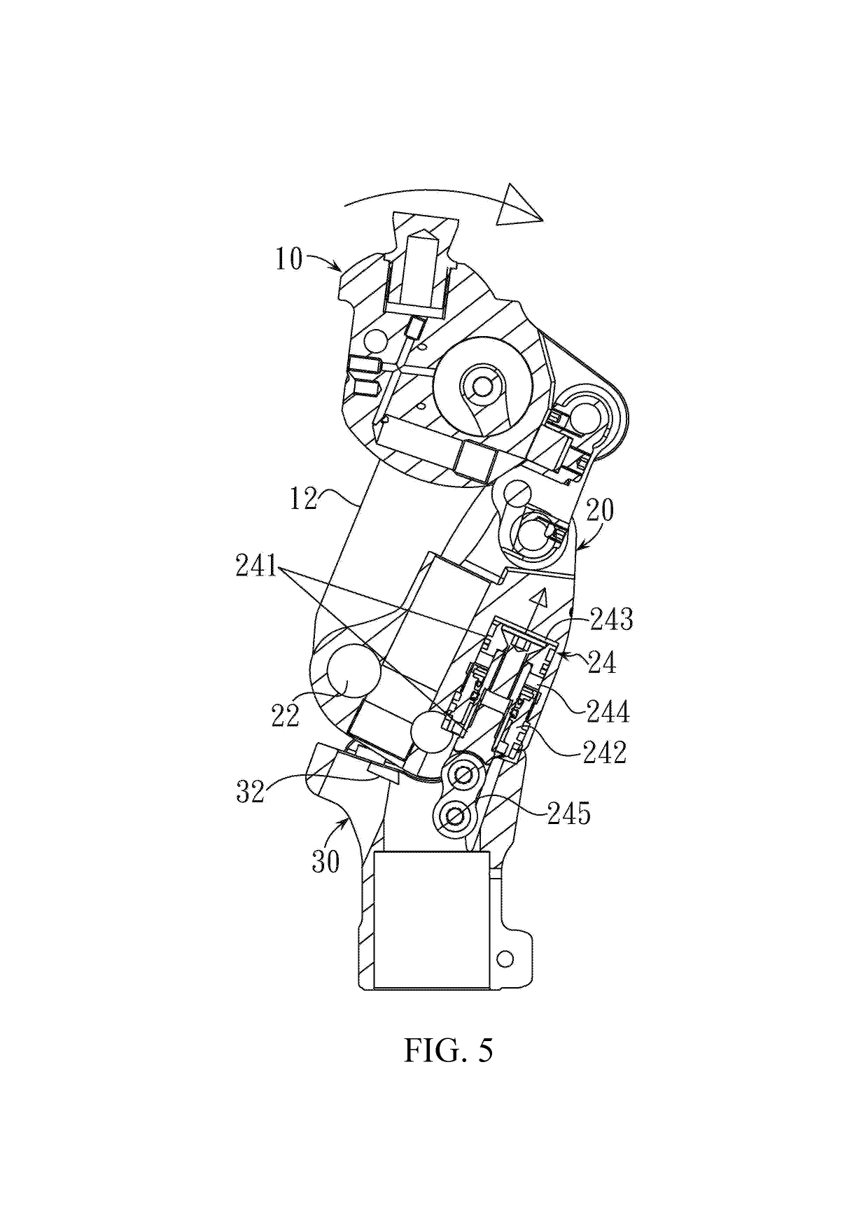

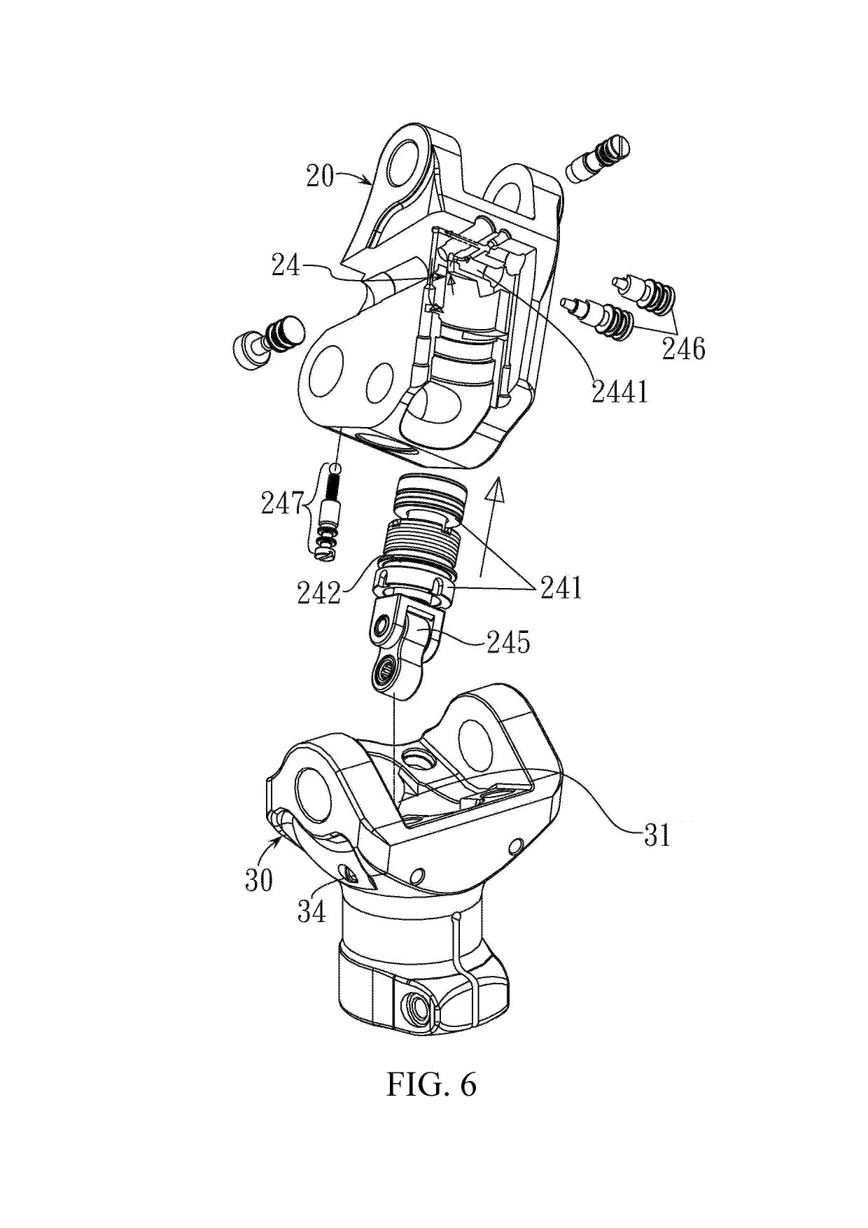

Subsequently referring to FIGS. 5-6, when the knee top seat (10) performs a backward tilting operation, fluid in the upper fluid compartment (243) inside the hydraulic cylinder (20) is driven to follow a backward-tilting hydraulic circuit (2441) for flowing as indicated by the arrow, so as to cushion the acting force generated by the backward tilting operation.

Further, a pair of valve needles (246) are arranged on a lateral side of the hydraulic cylinder (20), and the valve needles (246) are arranged at locations for respectively extending into the frontward-tilting hydraulic circuit (2431) and the backward-tilting hydraulic circuit (2441), so that through adjustment made with rotation of the valve needles (246), the flowrates in the two hydraulic circuits can be controlled in order to adjust the strength level of the cushioning. The frontward-tilting hydraulic circuit (2431) and the backward-tilting hydraulic circuit (2441) are each provided with at least one one-way valve (247) to stably keep the flowing direction in each of the hydraulic circuits and also to make the effect of cushioning more stable.

A cushion body (30) is connected to the hydraulic cylinder (20) by means of the fixing axles (25). The cushion body (30) is provided, on an upper side thereof, with a plurality of elastic elements (32) that provide an effect of cushioning to the acting force generated by the hydraulic cylinder (20). The upper side of the cushion body (30) is formed with a cushion-body through hole (31). The cushion-body through hole (31) is primarily provided for mounting of a push bar (245) connected to a bottom of the piston assembly (241). The cushion body (30) is formed, in a lateral side thereof, with a cushion-body pin hole (33) for receiving a fixing pin (34) to penetrate through the cushion-body pin hole (33) and the push bar (245) to thereby limit a displacement range of the push bar (245) such that adjustment can be made for a cushioning range of frontward tilting of 5 degrees and backward tilting of 5 degrees when the hydraulic cylinder (20) and the push bar (245) are mounted, and thus, making the adjustment of the entire knee joint with respect to cushioning and shock absorbing more flexible.

In brief, the present invention provides a standing cushioning adjustment. structure of a knee joint, in which in addition to the link bar (12) making linking to improve structural stability, adjusting of the frontward-tilting hydraulic circuit (2431) and the backward-tilting hydraulic circuit (2441) in the hydraulic cylinder (20) by means of the valve needles (246) enables adjustment of the strength of cushioning provided by the hydraulic cylinder (20), and the push bar (245) allows a force generated through operation of the hydraulic chamber (24) to completely transmit to the cushion body (30) to be thereby cancelled, allowing the hydraulic cylinder (20) and the cushion body (30) to operate in a range of 5 degrees in frontward tilting and backward tilting as being constrained structurally and also allowing an acting force generated through operation to be subjected to cushioning in a smooth and stable manner, so as to provide a user with bettered feedback adaptability for walk through frontward/backward tilting.

I claim:

1. A knee joint cushioning adjustment structure, comprising: a knee top seat, which comprises a first connecting hole and a link bar; a hydraulic cylinder, which comprises an upper part in which a second connecting hole is formed and a lower part in which a third connecting hole is formed, and a link-bar through hole formed between the second connecting hole and the third connecting hole, the first connecting hole being arranged at an inner side of the second connecting hole for connection therewith through a fixing axle, the link bar being connected to the link-bar through hole, a hydraulic chamber being formed in an interior of the hydraulic cylinder, wherein a piston assembly is arranged in the hydraulic chamber and penetrates through a hydraulic cylinder cap to define an upper fluid compartment and a lower fluid compartment, wherein during frontward tilting, the knee top seat drives the lower fluid compartment of the hydraulic cylinder to operate in combination with a frontward-tilting hydraulic circuit to implement cushioning, and during backward tilting, the knee top seat drives the upper fluid compartment of the hydraulic cylinder to operate in combination with a backward-tilting hydraulic circuit to implement cushioning; and a cushion body, which comprises a cushion-body through hole formed in an upper side thereof to receive a push bar connected to a bottom of the piston assembly to mount therein, the cushion body having a lateral side in which a cushion-body pin hole is formed to receive a fixing pin to penetrate therein and constrain the push bar, so as to provide, when mounted with the hydraulic cylinder and the push bar, a safe cushioning angle for frontward tilting and backward tilting to make an effect of cushioning and shock absorbing significant.

2. The knee joint cushioning adjustment structure according to claim 1, wherein a pair of valve needles are arranged on a lateral side of the hydraulic cylinder, and the valve needles are set at locations to respectively extend into the frontward-tilting hydraulic circuit and the backward-tilting hydraulic circuit for rotation and adjustment of flowrates in the hydraulic circuits to adjust strength of cushioning.

3. The knee joint cushioning adjustment structure according to claim 2, wherein the frontward-tilting hydraulic circuit and the backward-tilting hydraulic circuit are each provided with at least one one-way valve to stably keep a flowing direction in each of the hydraulic circuits and also to stabilize a function of cushioning.
